(19) United States
(12) Patent Application Publication
(10) Pub. No.: US 2026/0207521 A1
(43) Pub. Date: Jul. 23, 2026

(54) METHOD FOR PREPARING CELL MEMBRANE BIOMIMETIC DRUG DELIVERY SYSTEM TARGETING ATHEROSCLEROTIC LESIONS, AND PRODUCT AND USE THEREOF

(71) Applicant: The Third Affiliated Hospital, Guangzhou Medical University (Guangzhou Medical Center for Critical, Liwan District Guangzhou (CN)

(72) Inventors: Jionghua HUANG, Liwan District Guangzhou (CN); Jiayi YIN, Liwan District Guangzhou (CN); Jinzhu HUANG, Liwan District Guangzhou (CN)

(21) Appl. No.: 19/452,013

(22) Filed: Jan. 16, 2026

Related U.S. Application Data
(63) Continuation of application No. PCT/CN2025/078516, filed on Feb. 21, 2025.

(30) Foreign Application Priority Data
Jan. 23, 2025 (CN) 202510112295.X

(51) Int. Cl. A61K 9/50; A61K 9/51; C12N 5/00; C12N 5/0786

(52) U.S. Cl. CPC A61K 9/5068; A61K 9/5089; A61K 9/5153; A61K 9/5192; C12N 5/0018; C12N 5/0645; C12N 2501/734

(57) ABSTRACT

A method for preparing a cell membrane biomimetic drug delivery system targeting atherosclerotic lesions, and a product and the use thereof. The method includes the steps of: first culturing macrophages, extracting and purifying macrophage membranes, adding an enzyme inhibitor complex, and storing the mixture; subjecting the stored macrophage membranes to repeated extrusion using a mini extruder with a nanopore membrane to obtain macrophage membrane vesicles; preparing PEG-PLGA-based drug-loaded nanoparticles using a solvent evaporation method; and finally, injecting the macrophage membrane vesicles and drug-loaded nanoparticles through two inlets of a microfluidic electroporation chip, respectively, applying an alternating electric field for treatment to obtain a uniformly dispersed mixture, and then co-extruding the mixture using a mini extruder with a nanopore membrane.

### CROSS-REFERENCE TO RELATED APPLICATION

This application is a continuation of International Patent Application No. PCT/CN2025/078516, filed on Feb. 21, 2025, which claims priority of the Chinese Patent Application No. 202510112295.X, filed on Jan. 23, 2025, both of which are incorporated by references in their entities.

### TECHNICAL FIELD

The present disclosure relates to the technical field of cell membrane biomimetic drug delivery, and in particular to a method for preparing a cell membrane biomimetic drug delivery system targeting atherosclerotic lesions, and a product and the use thereof.

### BACKGROUND

Atherosclerosis is a typical chronic inflammatory disease of the vascular wall, where rupture of the vulnerable plaque causes diseases such as stroke, heart failure, peripheral vascular disease and ischemic heart disease, with mortality remaining at a high level, which poses a severe threat to human life and health.

Conventional anti-inflammatory and lipid-lowering drugs are commonly used in the clinic to treat atherosclerosis. However, these small molecule drugs often circulate in vivo for short periods of time and have toxic and side effects, resulting in low drug utilization and unsatisfactory therapeutic efficacy. In recent years, the biomimetic strategy of using cell membrane-coated nanoparticles has made significant progress in targeted drug delivery studies for a variety of diseases. Cell membrane-coated nanoparticles are a novel drug delivery carrier and biomimetic nanomaterial prepared by coating the surface of nanoparticles with cell membrane. Currently, methods for coating cell membrane nanoparticles include physical extrusion methods and ultrasound treatment methods. Although both of the above methods are effective in preparing membrane biomimetic nanoparticles, the physical extrusion method is time-consuming and labor-intensive, while the ultrasound treatment method tends to damage the core nanoparticles. Therefore, in the preparation of cell membrane biomimetic drug delivery system, how to develop an efficient method for preparing membrane biomimetic nanoparticles displays excellent research value and broad development prospects.

### SUMMARY

In view of the problems in the prior art, the present disclosure provides a method for preparing a cell membrane biomimetic drug delivery system targeting atherosclerotic lesions, and a product and use thereof.

In order to achieve the above object, the present disclosure provides the following technical solutions:

a method for preparing a cell membrane biomimetic drug delivery system targeting atherosclerotic lesions, including steps of:
a) culturing macrophages, extracting and purifying macrophage membranes, adding an enzyme inhibitor complex to the macrophage membranes, and storing the macrophage membranes at a temperature of 2-4° C.;
b) subjecting the stored macrophage membranes to repeated extrusion by using a mini extruder with a nanopore membrane to obtain macrophage membrane vesicles;
c) preparing PEG-PLGA-based drug-loaded nanoparticles using a solvent evaporation method; and
d) finally, injecting the macrophage membrane vesicles and drug-loaded nanoparticles through two inlets of a microfluidic electroporation chip at a mass ratio of (9-10):1, respectively, applying an alternating electric field for treatment for 90-120 s to obtain a uniformly dispersed mixture of macrophage membrane vesicles and drug-loaded nanoparticles, and then co-extruding the mixture using a mini extruder with a nanopore membrane, whereby a well-dispersed cell membrane biomimetic drug delivery system targeting atherosclerotic lesions may be finally prepared.

As a further technical solution, the enzyme inhibitor complex in step a) is prepared by mixing a protease inhibitor and a phosphatase inhibitor at a mass ratio of (2-3):1.

As a further technical solution, a method for preparing the PEG-PLGA drug-loaded nanoparticles in step c) is as follows: 10-15 mL of ultrapure water containing 2-2.5% (v/v) Tween 80 (polyoxyethylene (20) sorbitan monooleate) is placed in a constant-temperature magnetic stirrer and stirred to obtain an aqueous solution; 100 mg of PEG-PLGA and 5 mg of rapamycin are then added to 5-7 mL of acetone, and the mixture is mixed uniformly by continuous shaking using a vortex mixer to obtain an organic phase mixture; the organic phase mixture is added dropwise to the above aqueous solution, stirring is continued, then the mixture is transferred to an ultrasonic cell disrupter for ultrasonic dispersion, after which the dispersion is treated with a rotary evaporator to completely remove acetone to obtain a colloidal solution, and finally, the colloidal solution is centrifuged, and the supernatant is collected to remove free drugs, and then subjected to three cycles of ultracentrifugation, washing and concentration using an ultrafiltration tube to obtain the product.

As a further technical solution, the rotary evaporator has a rotation speed of 120-150 rpm, an evaporation temperature of 40-43° C., and a processing time of 100-120 min.

As a further technical solution, the alternating electric field in step d) has a frequency of 40-45 kHz and a field strength of 2.2-2.8 kV/cm.

As a further technical solution, the nanopore membranes inside the mini extruder in step b) and step d) are both used in order of 1 μm, 350 nm and 180 nm.

As a further technical solution, provided is a cell membrane biomimetic drug delivery system targeting atherosclerotic lesions prepared by the preparation method.

As a further technical solution, provided is use of the cell membrane biomimetic drug delivery system in the preparation of an anti-atherosclerotic drug-loaded system.

Compared with the conventional technology, the present disclosure has the following beneficial effects:
the cell membrane biomimetic drug delivery system prepared by the present disclosure may fully inherit the membrane surface functions of source macrophages, has the advantages of biodegradability, sustained drug release, long-lasting circulation, structural stability, and active/passive dual high-efficiency targeting, and may effectively inhibit the progression of atherosclerotic plaques, thereby achieving safe, stable and highly efficient treatment of atherosclerosis.

In the preparation process of the present disclosure, an enzyme inhibitor complex is added after the extraction and purification of the macrophage membranes, so that the biological activity of the macrophage membrane proteins may be maintained and the storage time thereof may be extended. Moreover, the coating of nanoparticles with macrophage membranes is achieved by means of microfluidic electroporation technology. An applied electric field induces the formation of reversible pores on the surface of macrophage membranes, enabling the introduction of exogenous molecules. This effectively facilitates the entry of PEG-PLGA drug-loaded nanoparticles into membrane vesicles. As a result, the cell membrane biomimetic drug delivery system has a uniform particle size in aqueous solution, maintains good dispersion characteristics, and exhibits no significant aggregation among particles.

### DETAILED DESCRIPTION OF THE EMBODIMENTS

The technical solutions in the examples of the present disclosure will be described clearly and completely below. Apparently, the described examples are only some of, rather than all of, the examples of the present disclosure. On the basis of the examples of the present disclosure, all other examples that may be obtained by those of ordinary skill in the art without creative efforts shall fall within the scope of protection of the present disclosure.

The following are specific examples:

### Example 1

A method for preparing a cell membrane biomimetic drug delivery system targeting atherosclerotic lesions, the method included steps of:
a) macrophages were cultured, macrophage membranes were extracted and purified, an enzyme inhibitor complex was added, and the macrophage membranes were stored at a temperature of 3° C., where the RAW264.7 macrophage line was used as a source of macrophage membranes, and the culture medium used was high-glucose DMEM medium containing 10% fetal bovine serum and 1% penicillin-streptomycin dual antibiotics;
b) the stored macrophage membranes were repeatedly extruded using a mini extruder with a nanopore membrane to obtain macrophage membrane vesicles;
c) PEG-PLGA-based drug-loaded nanoparticles were prepared by using a solvent evaporation method; and
d) finally, the macrophage membrane vesicles and drug-loaded nanoparticles were injected through two inlets of a microfluidic electroporation chip at a mass ratio of 10:1, respectively, an alternating electric field was applied for treatment for 110 s to obtain a uniformly dispersed mixture of macrophage membrane vesicles and drug-loaded nanoparticles, and then the mixture was co-extruded by using a mini extruder with a nanopore membrane, whereby a well-dispersed cell membrane biomimetic drug delivery system targeting atherosclerotic lesions was finally prepared.

In the example: the enzyme inhibitor complex was prepared by mixing a protease inhibitor and a phosphatase inhibitor at a mass ratio of 3:1; a method for preparing the PEG-PLGA drug-loaded nanoparticles in step c) was as follows: 12 mL of ultrapure water containing 2.3% (v/v) Tween 80 (polyoxyethylene (20) sorbitan monooleate) was placed in a constant-temperature magnetic stirrer and stirred to obtain an aqueous solution; 100 mg of PEG-PLGA and 5 mg of rapamycin were then added to 6 mL of acetone, and the mixture was mixed uniformly by continuous shaking using a vortex mixer to obtain an organic phase mixture; the organic phase mixture was added dropwise to the above aqueous solution, stirring was continued, then the mixture was transferred to an ultrasonic cell disrupter for ultrasonic dispersion, after which the dispersion was treated with a rotary evaporator to completely remove acetone to obtain a colloidal solution, and finally, the colloidal solution was centrifuged, and the supernatant was collected to remove free drugs, and then subjected to three cycles of ultracentrifugation, washing and concentration using an ultrafiltration tube to obtain the product; the rotary evaporator had a rotation speed of 130 rpm, an evaporation temperature of 42° C., and a processing time of 110 min; the alternating electric field in step d) had a frequency of 43 kHz and a field strength of 2.6 kV/cm.

### Example 2

Example 2 was essentially the same as Example 1 except that: the enzyme inhibitor complex in step a) was prepared by mixing a protease inhibitor and a phosphatase inhibitor at a mass ratio of 2:1, and the enzyme inhibitor complex was stored at a temperature of 2° C.; a method for preparing the PEG-PLGA drug-loaded nanoparticles in step c) was as follows: 10 mL of ultrapure water containing 2% (v/v) Tween 80 (polyoxyethylene (20) sorbitan monooleate) was placed in a constant-temperature magnetic stirrer and stirred to obtain an aqueous solution; 100 mg of PEG-PLGA and 5 mg of rapamycin were then added to 5 mL of acetone, and the mixture was mixed uniformly by continuous shaking using a vortex mixer to obtain an organic phase mixture; the organic phase mixture was added dropwise to the above aqueous solution, stirring was continued, then the mixture was transferred to an ultrasonic cell disrupter for ultrasonic dispersion, after which the dispersion was treated with a rotary evaporator to completely remove acetone to obtain a colloidal solution, and finally, the colloidal solution was centrifuged, and the supernatant was collected to remove free drugs, and then subjected to three cycles of ultracentrifugation, washing and concentration using an ultrafiltration tube to obtain the product; the rotary evaporator had a rotation speed of 120 rpm, an evaporation temperature of 40° C., and a processing time of 100 min; in step d), the macrophage membrane vesicles and drug-loaded nanoparticles were injected through two inlets of a microfluidic electroporation chip at a mass ratio of 9:1, respectively, and then an alternating electric field was applied for treatment for 90 s; the alternating electric field had a frequency of 40 kHz and a field strength of 2.2 kV/cm.

### Example 3

Example 2 was essentially the same as Example 1 except that: the enzyme inhibitor complex in step a) was prepared by mixing a protease inhibitor and a phosphatase inhibitor at a mass ratio of 3:1, and the enzyme inhibitor complex was stored at a temperature of 4° C.; a method for preparing the PEG-PLGA drug-loaded nanoparticles in step c) was as follows: 15 mL of ultrapure water containing 2.5% (v/v) Tween 80 (polyoxyethylene (20) sorbitan monooleate) was placed in a constant-temperature magnetic stirrer and stirred to obtain an aqueous solution; 100 mg of PEG-PLGA and 5 mg of rapamycin were then added to 7 mL of acetone, and the mixture was mixed uniformly by continuous shaking using a vortex mixer to obtain an organic phase mixture; the organic phase mixture was added dropwise to the above aqueous solution, stirring was continued, then the mixture was transferred to an ultrasonic cell disrupter for ultrasonic dispersion, after which the dispersion was treated with a rotary evaporator to completely remove acetone to obtain a colloidal solution, and finally, the colloidal solution was centrifuged, and the supernatant was collected to remove free drugs, and then subjected to three cycles of ultracentrifugation, washing and concentration using an ultrafiltration tube to obtain the product; the rotary evaporator had a rotation speed of 150 rpm, an evaporation temperature of 43° C., and a processing time of 120 min; in step d), the macrophage membrane vesicles and drug-loaded nanoparticles were injected through two inlets of a microfluidic electroporation chip at a mass ratio of 10:1, respectively, and then an alternating electric field was applied for treatment for 120 s; the alternating electric field had a frequency of 45 kHz and a field strength of 2.8 kV/cm.

Comparative Example 1: This comparative example was essentially the same as Example 1 except that: the macrophage membrane vesicles and drug-loaded nanoparticles in step d) were treated with ultrasound (42 kHz, 100 W) in ice water for 2 min using an ultrasonic apparatus at a mass ratio of 10:1, to obtain a uniformly dispersed mixture of macrophage membrane vesicles and drug-loaded nanoparticles, and the mixture was then co-extruded using a mini extruder with a nanopore membrane.

Test: In order to verify the size, uniformity and surface potential characteristics of the cell membrane biomimetic drug delivery system, the mean particle size, PDI dispersion coefficient and Zeta potential of the biomimetic nanoparticles were determined using a dynamic light scattering instrument. The results are as shown in Table 1.

TABLE 1

| | Mean particle | PDI dispersion | Zeta |
|---|---|---|---|
| | size (nm) | coefficient | potential (mv) |
| Example 1 | 125.13 | 0.095 | −19.5 |
| Example 2 | 127.40 | 0.101 | −20.1 |
| Example 3 | 128.64 | 0.103 | −19.8 |
| Comparative | 131.85 | 0.115 | −20.8 |
| Example 1 | | | |

As can be seen from Table 1, the cell membrane biomimetic drug delivery system targeting the atherosclerotic lesion prepared in the present disclosure has a uniform particle size in aqueous solution, maintains good dispersion characteristics, and exhibits no significant aggregation among particles.

The above preferred examples of the present disclosure are merely intended to illustrate the present disclosure. The preferred examples neither describe all details elaborately, nor limit the present disclosure to be merely the specific implementation. Apparently, many modifications and changes may be made according to the contents of this specification.

What is claimed is:

1. A method for preparing a cell membrane biomimetic drug delivery system targeting atherosclerotic lesions, comprising steps of:
a) culturing macrophages, extracting and purifying macrophage membranes, adding an enzyme inhibitor complex to the macrophage membranes, and storing the macrophage membranes at a temperature of 2-4° C.;
b) subjecting the stored macrophage membranes to repeated extrusion by using a mini extruder with a nanopore membrane to obtain macrophage membrane vesicles;
c) preparing PEG-PLGA-based drug-loaded nanoparticles by using a solvent evaporation method; and
d) injecting the macrophage membrane vesicles and drug-loaded nanoparticles through two inlets of a microfluidic electroporation chip at a mass ratio of (9-10):1, respectively, applying an alternating electric field for treatment for 90-120 s to obtain a mixture of macrophage membrane vesicles and drug-loaded nanoparticles, and then co-extruding the mixture of macrophage membrane vesicles and drug-loaded nanoparticles by using a mini extruder with a nanopore membrane, whereby a cell membrane biomimetic drug delivery system targeting atherosclerotic lesions is obtained.

2. The method for preparing the cell membrane biomimetic drug delivery system targeting the atherosclerotic lesions according to claim 1, wherein the enzyme inhibitor complex in step a) is prepared by mixing a protease inhibitor and a phosphatase inhibitor at a mass ratio of (2-3):1.

3. The method for preparing the cell membrane biomimetic drug delivery system targeting the atherosclerotic lesions according to claim 1, wherein a method for preparing the PEG-PLGA drug-loaded nanoparticles in step c) is as follows: stirring 10-15 mL of ultrapure water containing 2-2.5% (v/v) polyoxyethylene (20) sorbitan monooleate in a constant-temperature magnetic stirrer to obtain an aqueous solution; adding 100 mg of PEG-PLGA and 5 mg of rapamycin to 5-7 mL of acetone to mix by continuous shaking using a vortex mixer to obtain an organic phase mixture; adding dropwise the organic phase mixture to the aqueous solution, stirring is continued, then the mixture of the organic phase mixture and the aqueous solution is transferred to an ultrasonic cell disrupter for ultrasonic dispersion, treating the dispersion with a rotary evaporator to completely remove acetone to obtain a colloidal solution, and centrifuging the colloidal solution and collecting the supernatant to remove free drugs, and then subjecting to three cycles of ultracentrifugation, washing and concentration by using an ultrafiltration tube to obtain the product.

4. The method for preparing the cell membrane biomimetic drug delivery system targeting the atherosclerotic lesions according to claim 3, wherein the rotary evaporator has a rotation speed of 120-150 rpm, an evaporation temperature of 40-43° C., and a processing time of 100-120 min.

5. The method for preparing the cell membrane biomimetic drug delivery system targeting the atherosclerotic lesions according to claim 1, wherein the alternating electric field in step d) has a frequency of 40-45 kHz and a field strength of 2.2-2.8 kV/cm.

6. The method for preparing the cell membrane biomimetic drug delivery system targeting the atherosclerotic lesions according to claim 1, wherein the nanopore membranes inside the mini extruder in step b) and step d) are both used in order of 1 μm, 350 nm and 180 nm.

7. A cell membrane biomimetic drug delivery system targeting atherosclerotic lesions prepared by the preparation method according to claim 1.

8. A method for preventing or treating atherosclerosis, wherein comprising administering the cell membrane biomimetic drug delivery system of claim 7 to a subject in need thereof.

9. A cell membrane biomimetic drug delivery system targeting atherosclerotic lesions prepared by the preparation method according to claim 2.

10. A cell membrane biomimetic drug delivery system targeting atherosclerotic lesions prepared by the preparation method according to claim 3.

11. A cell membrane biomimetic drug delivery system targeting atherosclerotic lesions prepared by the preparation method according to claim 4.

12. A cell membrane biomimetic drug delivery system targeting atherosclerotic lesions prepared by the preparation method according to claim 5.

13. A cell membrane biomimetic drug delivery system targeting atherosclerotic lesions prepared by the preparation method according to claim 6.

14. A method for preventing or treating atherosclerosis, wherein comprising administering the cell membrane biomimetic drug delivery system of claim 9 to a subject in need thereof.

15. A method for preventing or treating atherosclerosis, wherein comprising administering the cell membrane biomimetic drug delivery system of claim 10 to a subject in need thereof.

16. A method for preventing or treating atherosclerosis, wherein comprising administering the cell membrane biomimetic drug delivery system of claim 11 to a subject in need thereof.

17. A method for preventing or treating atherosclerosis, wherein comprising administering the cell membrane biomimetic drug delivery system of claim 12 to a subject in need thereof.

18. A method for preventing or treating atherosclerosis, wherein comprising administering the cell membrane biomimetic drug delivery system of claim 13 to a subject in need thereof.
